(19) United States
(12) Patent Application Publication
(10) Pub. No.: US 2003/0180219 A1
(43) Pub. Date: Sep. 25, 2003

(54) Mycoplasma bovis challenge model and methods for administering M.bovis and methods for inducing pneumonic lung lesions

(75) Inventors: Robin Lee Keich, Waterford, CT (US); Laszlo Pal Stipkovits, Budapest (HU)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/227,730

(22) Filed: Aug. 26, 2002

Related U.S. Application Data
(60) Provisional application No. 60/315,324, filed on Aug. 28, 2001.

(51) Int. Cl. A61K 49/00; A61K 39/02

(52) U.S. Cl. 424/9.2; 424/264.1

(57) ABSTRACT

The present invention provides a reproducible M. bovis challenge model and methods for reliably inducing and establishing a disease or disorder caused by infection with M. bovis by administering to an animal an effective amount of a M. bovis culture. The challenge culture of the present invention is administered in an amount sufficient to elicit M. bovis specific cellular or humoral immune responses. The M. bovis challenge model in accordance with the present invention can be used to evaluate the efficacy of potential vaccines.

### FIELD OF THE INVENTION

This invention relates to a Mycoplasma bovis challenge model method and methods for administering M. Bovis, inducing, and establishing a disease or disorder in an animal caused by M. bovis. The M. bovis challenge model in accordance with the present invention can be used to evaluate the efficacy of potential vaccines.

### BACKGROUND OF THE INVENTION

Mycoplasma bovis is an important global bovine pathogen in housed or intensively reared beef and dairy cattle. The most frequently reported clinical manifestation is pneumonia of calves, which is often accompanied by arthritis, also known as pneumonia-arthritis syndrome. Its etiological role has also been associated with mastitis, otitis, and reproductive disease or disorders of cows and bulls. Significant economic losses are linked with M. bovis induced respiratory disease, since M. bovis has been associated with up to 36% of the mortality due to bovine respiratory disease (BRD). In order to reduce mortality, antibiotic therapy is often used since no fully licensed M. bovis vaccines are currently available. Prevention of M. bovis disease may also reduce predisposition of the animal to other respiratory diseases. A M. bovis bacterin that is highly efficacious and safe for young calves would be very valuable to the cattle industry. Therefore, a reproducible M. bovis challenge model that induces significant lung lesions and other clinical signs is needed to evaluate the efficacy of potential M. bovis vaccines.

### SUMMARY OF THE INVENTION

The present invention provides a reproducible M. bovis challenge model and methods for reliably inducing and establishing a disease or disorder caused by infection with M. bovis by administering to an animal an effective amount of a M. bovis culture. The challenge culture of the present invention is administered in an amount sufficient to elicit M. bovis specific cellular or humoral immune responses. In one aspect, the animal is a calf. The M. bovis challenge model in accordance with the present invention can be used to evaluate the efficacy of potential vaccines.

The invention also provides a method for the preparation of a M. bovis challenge culture, which comprises growing an isolate of M. bovis in culture in a suitable medium to a sufficient viable count and a method for experimentally administering the culture to an animal to induce pneumonic lesions and clinical signs of disease.

### BRIEF DESCRIPTION OF THE DRAWINGS

FIG. 1 is a graph showing group mean body temperatures prior to and following experimental M. bovis challenge. M. bovis challenged calves (Treatment Groups A and C) had higher mean body temperatures on days 1 through 20 when compared to the non-challenged control animals (Treatment Group D). Group B challenged calves had higher mean body temperatures on days 3 through 5, days 7 through 14, and day 17 when compared to Group D (non-challenged control animals).

FIG. 2 is a graph showing group mean body temperatures prior to and following experimental M. bovis challenge. M. bovis challenged calves (Treatment Group A) had higher mean body temperatures on days 15, 17, and 18 when compared to the non-challenged control animals (Treatment Group B).

FIG. 3 is a graph showing group mean body temperatures prior to and following experimental M. bovis challenge. M. bovis challenged calves (Treatment Groups B and C) had higher mean body temperatures on days 4 through 21 when compared to the non-challenged control animals (Treatment Group D). Group A challenged calves had higher mean body temperatures on days 7 through 21 when compared to Group D (non-challenged control animals).

FIG. 4 is a graph showing group mean body temperatures from 3 days to 20 days following experimental M. bovis challenge. Calves administered two doses of the M. bovis vaccines (Treatment Groups A and C) and animals in Group E (non-challenged control animals) had lower mean body temperatures on days 7 through 20 when compared to the placebo challenged group (Treatment Group D). Vaccinated calves in treatment Group B had lower mean body temperatures on days 7 to 12 and days 14 to 20 when compared to the placebo challenged group (Treatment Group D).

### DETAILED DESCRIPTION OF THE INVENTION

The present invention encompasses a challenge model and method of inducing a disease or disorder in an animal caused by infection with M. bovis comprising administering to the animal an effective amount of a M. bovis culture. The invention encompasses methods of preparing and administering a M. bovis culture. Examples of M. bovis strains are ATCC 25025 (deposited by R. G. Wittier on Oct. 8, 1968), 25523 (deposited by R. G. Wittier on Oct. 22, 1969) and 27368 (deposited by R. G. Wittier on Jul. 5, 1972). In a preferred embodiment, the M. bovis isolate of the challenge culture comprises one or more of the following strains: 2300 (ATCC PTA-3558), 3625 (ATCC PTA-3559), 16150 (ATCC PTA-3560), 20518 (ATCC PTA-3561), or 5063.

The present invention contemplates that any M. bovis isolate can be used as an effective challenge culture. In a preferred embodiment, the M. bovis isolates are grown in Beg4 medium to a sufficient cell density and an effective amount is administered to an animal to induce pneumonic lesions and clinical signs of disease. The deposit of the M. bovis strains 2300 (ATCC PTA-3558), 3625 (ATCC PTA-3559), 16150 (ATCC PTA-3560), 20518 (ATCC PTA-3561), and 5063 isolate was made pursuant to the Budapest Treaty on the International Recognition of the Deposit of Microorganisms for the Purpose of Patent Procedure, with the American Type Culture Collection, 10801 University Boulevard, Manassas, Va. 20110-2209.

For clarity of disclosure, and not by way of limitation, the detailed description of the invention is divided into the following subsections which describe or illustrate certain features, embodiments or applications of the invention.

### Definitions And Abbreviations

The abbreviation M., preceding the name of a species, refers to the genus Mycoplasma.

The term “disease or disorder” with respect to a M. bovis infection as used herein means to cause the replication of the M. bovis bacteria, to induce M. bovis shedding or transmission, or to establish a M. bovis infection in its host, and to cause symptoms of a M. bovis infection. The challenge model is considered effective if there is an increase in bacterial load, increase in pulmonary infections, increases in lung lesions, increased clinical signs, i.e. increased rectal temperatures and/or decreases in weight gain and/or growth. The method of the present invention is, for example, effective in inducing pneumonia, respiratory infections and lung lesions, increasing the level of M. bovis in the lung, increasing temperatures, and decreasing weight gains in animals and especially cattle. The present invention also contemplates that the administration of an effective amount of a M. bovis challenge culture to animals, and preferably cattle will induce disorders including pneumonia, arthritis, mastitis, otitis and reproductive disorders in such animals.

The term “M. bovis challenge culture” as used herein refers to a culture useful in creating a disorder or disease caused by M. bovis infection. The M. bovis culture can include any culture effective in causing infection in cattle by M. bovis. The M. bovis culture that may be used in the present invention can include, for example, a fresh, frozen or lyophilized M. bovis cell preparation.

The term “M. bovis challenge model” as used herein refers to a method of administering a M. bovis culture that is useful in creating a disorder or disease caused by M. bovis infection. The M. bovis challenge model can include any method of administration that is effective in causing infection in cattle by M. bovis. The method of administration that may be used in the present invention can include, for example, oral, intranasal, oranasal, topical, transdermal, aerosol, and parenteral (e.g., intravenous, intraperitoneal, intratracheal, intradermal, subcutaneous or intramuscular).

The term “animal” as used herein refers to all non-human animals, including mammals.

The term “cattle” as used herein refers to bovine animals including but not limited to steer, bulls, cows, and calves. Preferably, the method of the present invention is applied to an animal which is a non-human mammal; most preferably, a calf.

The term “effective amount” refers to an amount of M. bovis culture sufficient to induce or establish disease in the subject to which it is administered. An effective amount of M. bovis challenge culture means, for example, that the challenge culture causes mycoplasmal pneumonia.

The term “M. bovis vaccine” as used herein refers to a vaccine useful in prevention or treating a disorder or disease caused by infection by M. bovis. M. bovis vaccine can include any vaccine effective in treating or preventing infection in cattle by virulent M. bovis. The M. bovis vaccine that may be used in the present invention can include, for example, a whole or partial M. bovis cell preparation, inactivated or modified live vaccines, a subunit vaccine having one or more M. bovis derived polypeptides or proteins, or immunogenic fragments of such proteins or polypeptides, or one or more M. bovis genes or nucleic acids encoding for one or more M. bovis derived polypeptides or proteins, or immunogenic fragments thereof, and which genes or nucleic acids are capable of being expressed in vivo in cattle. The M. bovis polypeptides, proteins, immunogenic fragments of such polypeptides and proteins, or M. bovis genes or nucleic acids can be synthesized or recombinantly produced using techniques known in the art.

The term “adjuvant” as used herein, is a potentiator of the immune response. Suitable adjuvants may include, but are not limited to: mineral gels, e.g., aluminum hydroxide; surface active substances such as lysolecithin; glycosides, e.g., saponin derivatives such as Quil A or GPI-0100; cationic surfactants such as DDA, pluronic polyols; polyanions; non-ionic block polymers, e.g., Pluronic F-127 (B.A.S.F., USA); peptides; mineral oils, e.g. Montanide ISA-50 (Seppic, Paris, France), carbopol, Amphigen (Hydronics Omaha, Nebr. USA), Alhydrogel (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol, cytokines and combinations of adjuvants. The immunogen may also be incorporated into liposomes, or conjugated to polysaccharides and/or other polymers for use in a vaccine formulation.

### Challenge Culture

The invention provides a M. bovis challenge model and a method for preparing and administering a M. bovis challenge culture which comprises growing a isolate of M. bovis in culture in a suitable medium to a sufficient cell density; and a method for experimentally administering the culture to an animal to induce pneumonic lesions and clinical signs of disease. In one embodiment M. bovis is isolated from lung tissue. In another embodiment, M. bovis is isolated from lymph node tissue. A variety of media are well known in the art and include Friis, Beg4, Hayflick's, and MHP. In a preferred embodiment, the M. bovis isolate of the challenge culture comprises one or more of the following strains: 2300, 3625, 16150, 20518 or 5063.

The conditions under which the M. bovis isolate is grown may vary depending upon the composition of the medium and the specific isolate being grown. However the isolate is typically grown as follows. A frozen vial of the isolate is quickly thawed or a lyophilized vial is resuspended in 1 to 10 ml of Beg4 medium. Sterile Beg4 medium is then inoculated with a 0.1% to 30% of the M. bovis seed stock. The culture is then incubated at 30° C. to 40° C. for 12 hours to about 72 hours, measured from the time of incubation to the time of harvest. In a preferred embodiment, the M. bovis culture is grown at 37° C. for 24 hours to 48 hours. For each challenge day, separate challenge inoculums are prepared. On each day of challenge, the viable count, colony forming unit (CFU), of the challenge culture is determined by serial dilution in Beg4 medium and plating of each serial dilution on Heart Infusion Agar (HIA) agar plates. The HIA plates are then incubated at 37° C. for 48 to 168 hours, preferably 120 hours and number of colonies are determined for the viable count.

The resulting M. bovis challenge culture can be concentrated. Various methods are known in the art for concentrating such organisms. For example, the organisms can be concentrated by centrifugation, e.g. ultracentrifugation, or by filtration, e.g. ultrafiltration. The concentrated, M. bovis culture which results is then recovered using methods well known in the art. The challenge culture can also be produced by any of several modifications to the preceding method, which are readily known to the skilled artisan.

The M. bovis culture that can be used in the present invention can include, for example, a fresh, frozen or lyophilized M. bovis cell preparation.

M. bovis isolates can also be obtained directly from infected cattle lung lesions using known techniques. M. bovis isolates can also be obtained directly from the nasal cavity, trachea, lung lavage fluid, lymph nodes, liver, spleen, kidney, heart, blood, and joints of infected cattle using known techniques.

### Dosing, Modes of Administration and Treatment

According to the present invention, at least one dose of an effective amount of a M. bovis culture administered to an animal and preferably a calf of approximately three to twenty-eight weeks of age causes a M. bovis infection. Preferably, the M. bovis culture is administered on three consecutive days. The effective amount of a M. bovis challenge culture contains about 1×10⁶ to about 5×10¹¹ colony forming units (CFU) per challenge dose. Preferably, a M. bovis challenge culture that provides sufficient disease contains about 1×10⁸ to about 1×10¹¹ CFU/dose and more preferably, about 1×10¹⁰ to about 5×10¹⁰ CFU/dose. In accordance with the present invention, a M. bovis infection is reproducibly induced and clinical disease established in cattle in about 1 to 49 days. Preferably, a M. bovis clinical disease is reproducibly established in about 1 to 21 days and more preferably in about 1 to 14 days.

According to the present invention, the effective amount of the M. bovis challenge culture for administration is about 0.5 to about 30.0 ml, preferably about 5 ml to about 20 ml, and more preferably, about 10 to 12 ml.

In accordance with the present invention, administration can be achieved by known routes, including the oral, intranasal, oranasal, topical, transdermal, aerosol, and parenteral (e.g., intravenous, intraperitoneal, intratracheal, intradermal, subcutaneous or intramuscular). A preferred route of administration is intranasal administration.

The present invention also contemplates a single dose challenge method, which eliminates the necessity of administration of additional challenge doses to calves in order to generate M. bovis induced disease.

According to the present invention, the administration of an effective amount of a M. bovis challenge administered to calves from approximately three to twenty-eight weeks of age provides an effective respiratory infection, including pneumonia, increases the level of M. bovis in the lung, increases temperatures, and decreases weight gains. The amount of M. bovis in the challenge culture, i.e. from 1×10⁶ to about 5×10¹¹, has for the first time, been determined to reliably and reproducibly induce infection in cattle and establish clinical disease in about 1 to 49 days.

The present invention provides a method of administering a M. bovis infection in a calf comprising administering to the calf at least one dose, and preferably three challenge doses of the culture so as to cause a M. bovis infection in the calf. In a preferred embodiment, the challenge culture is administered intranasally. Moreover, it is preferred that the challenge dose comprise about 10 to 12 ml of the culture, each-ml containing about 1.0×10⁹ M. bovis colony forming units. The challenge culture is desirably administered to the calf on three consecutive days.

The present invention also contemplates that the administration of an effective amount of a M. bovis challenge culture administered to animals, and preferably cattle to cause disorders including, but not limited to pneumonia, arthritis, mastitis, otitis and reproductive disorders in such animals.

The present invention is further illustrated by the following examples.

### EXAMPLE 1

### Materials and Methods

Animals

Healthy crossbred dairy or beef calves were obtained for each experimental challenge. Calves were allowed to acclimate for a minimum of seven days prior to the initiation of each study. All calves received a concentrated non-medicated diet daily, free of any known contaminants or pesticides and had free access to water.

Challenge Method

Each calf received 10 to 20 ml of a fresh M. bovis culture [approximately 1×10⁸ to 1×10¹⁰ colony forming units (CFU/ml)] by the intranasal route on three consecutive days. A viable count (CFU/ml) of the challenge inoculum was determined shortly after the completion of each experimental challenge. The viable count of the challenge culture was determined by serial dilution in Beg4 medium and plating of each serial dilution on HIA agar plates. The HIA plates were then incubated at 37° C. for 120 hours and number of colonies were determined for the viable count.

Experimental Procedure

A unique ear tag number identified each calf. Animals were randomly assigned by age into pens and treatment groups.

All animals were weighed at 1 day prior to challenge, 7 days following challenge, 14 days following challenge, and at approximately 3 weeks following challenge.

Rectal temperatures were measured each morning 1 day prior to challenge, immediately prior to challenge, and for 20 days following challenge.

A blood sample was collected from each calf from the jugular vein. Calves were bled at approximately 1 day prior to challenge, 7 days following challenge, 14 days following challenge, and at necropsy (approximately 3 weeks post-challenge). Serum from each blood sample was stored at −20° C. until evaluated by a M. bovis ELISA kit (Chekit M. bovis Sero) prepared by Bommeli AG (Hoechst Roussel Vet Diagnostics, Liebefeld-Bern, Switzerland). The ELISA plates were read using a Multiscan reader at a wavelength of 405 nm. Optical density (OD) values were translated to a percentage relating to the OD value of the positive control serum, using the following formula: percentage=(Sample OD-Negative serum OD)/(positive serum OD-Negative serum OD)*100. Values lower than 60% were considered negative. Sera having percentages between 60 and 80% were considered suspect, while sera showing OD greater than 80% were accepted as positive.

All animals were necropsied at approximately 3 weeks following the experimental M. bovis challenge. Calves were euthanized and all major organs, excluding the central nervous system, were examined grossly.

Lungs were removed and evaluated grossly for characteristic lesions attributable to a M. bovis infection. Lesions were sketched on a standard lung diagram. Percent gross involvement per each lung lobe was weighted using the following ratios of individual lung lobes to total lung mass.

| Lung Lobe | Percentage of Lung |
|---|---|
| Left Apical | 5 |
| Right Apical | 6 |
| Middle | 5 |
| Left Cardiac | 6 |
| Right Cardiac | 7 |
| Accessory | 4 |
| Left Diaphragmatic | 32 |
| Right Diaphragmatic | 35 |

The weighted lung lobe values were then summed in order to determine the percentage of total lung with gross lesions (Pointon et al, 1992). In addition the following formula was used to calculate the percent reduction in lung damage (lesions).

$$100 - \frac{Mean Percent Lung Damage of Treatment Group}{Mean Percent Lung Damage of Control Group} = Percent Reduction$$

In addition, each lung was lavaged with 50 ml of PBS. Attempts were made to isolate and determine the viable M. bovis counts from the bronchial lavage fluid. The M. bovis viable count (CFU/ml) was determined by preparing appropriate serial dilutions of bronchial lavage fluid and plating samples onto an appropriate agar medium.

### EXAMPLE 2

In this example, different M. bovis challenge methods were evaluated in young calves. Twenty-four, healthy crossbred dairy calves (Holstein/Friesian cross), approximately 7 weeks of age, with no maternal antibody to M. bovis were obtained and were randomly assigned by age as shown in Table 1. All calves were allowed to acclimate for three weeks prior to the initiation of the study.

TABLE 1
Experimental Treatment Groups

| Treatment | | Number |
|---|---|---|
| Group | Treatment | of Animals |
| A | Aerosol Challenge (2 consecutive days) | 6 |
| B | Aerosol Challenge (3 consecutive days) | 6 |
| C | Intranasal Challenge (3 consecutive days) | 6 |
| D | Non-Challenged Controls | 6 |

Calves were challenged as described above at approximately ten weeks of age. Each calf received 10 ml (5 ml per nostril) of a fresh culture of M. bovis strain 5063 on either two or three consecutive days. Animals in Groups A and B were challenged with a broth culture by aerosol through a mask. Calves in Group C were challenged by using a simple spray device (Genesis Industries, Elmwood, Wis.) and animals in Group D were left as non-challenged control calves.

A viable count (CFU/ml) of the challenge inoculum was determined within one hour after the completion of each M. bovis experimental challenge. Results are shown in Table 2.

TABLE 2
Viable Count (CFU/ml) of Mycoplasma bovis Challenge Inoculum

| Challenge Culture | CFU/ml |
|---|---|
| Day 1 | 5.0 × 10⁸ |
| Day 2 | 1.0 × 10⁹ |
| Day 3 | 5.3 × 10⁸ |

All animals were weighed at 1 day prior to challenge, 7 days following challenge, 14 days following challenge, and 20 days following experimental M. bovis challenge. Results are summarized in Table 3. Calves that were administered the experimental M. bovis challenge (Treatment Groups A, B, and C) had decreased weight gains when compared to the non-challenged control animals (Treatment Group D).

TABLE 3
Summary of Body Weights Following Experimental Mycoplasma bovis Challenge
Mean Body Weight (kg) ± Standard Deviation

| Treatment | | | | | Weight |
|---|---|---|---|---|---|
| Group | Day −1 | Day 7 | Day 14 | Day 20 | Gain |
| A | 75.0 ± 10.0 | 76.8 ± 10.0 | 79.9 ± 8.3 | 82.8 ± 10.9 | 7.8 |
| B | 73.2 ± 5.9 | 77.2 ± 6.7 | 80.3 ± 10.3 | 88.2 ± 8.0 | 15.0 |
| C | 69.0 ± 3.9 | 74.7 ± 5.6 | 75.3 ± 5.7 | 85.3 ± 7.3 | 16.3 |
| D | 75.1 ± 7.5 | 79.2 ± 6.8 | 86.5 ± 8.6 | 105.5 ± 6.8 | 30.4 |

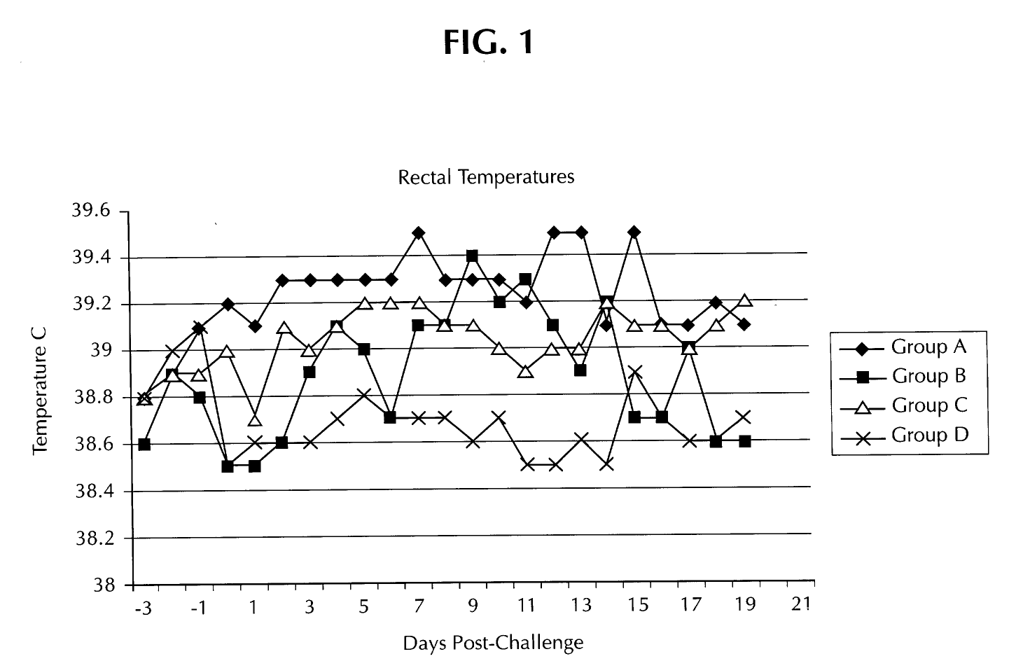

Rectal temperatures were measured each morning 3 days, 2 days and 1 day prior to challenge, immediately prior to challenge, and for 19 days following experimental M. bovis challenge. Results are summarized in FIG. 1. M. bovis challenged calves (Treatment Groups A and C) showed clinical signs, i.e. higher mean body temperatures on days 1 through 20 when compared to the non-challenged control animals (Treatment Group D). Group B challenged calves had higher mean body temperatures on days 3 through 5, days 7 through 14, and day 17 when compared to Group D (non-challenged control animals).

M. bovis specific serum antibody responses (IgG) are summarized in Table 4. Serum samples with percent optical density (OD) values>0.80 of the positive control serum were considered positive for M. bovis. All calves were M. bovis negative prior to experimental challenge. Calves that received the experimental M. bovis challenge (Treatment Groups A, B, and C) were seropositive on day 20 following the M. bovis challenge. Animals in Treatment Group D (non-challenged control animals) were essentially negative throughout this study.

TABLE 4
Summary of Mycoplasma bovis Serum Antibody (IgG)
Mean Percentage of Optical Density Values to Positive Control Serum
± Standard Deviation

| Treatment | | | | |
|---|---|---|---|---|
| Group | Day -1 | Day 7 | Day 14 | Day 20 |
| A | 55.9 ± 41.7 | 108.1 ± 64.2 | 108.1 ± 64.2 | 142.7 ± 17.1 |
| B | 52.7 ± 53.8 | 64.5 ± 39.7 | 64.5 ± 39.7 | 151.4 ± 10.3 |
| C | 69.8 ± 50.3 | 82.1 ± 42.1 | 82.1 ± 42.1 | 118.0 ± 33.0 |
| D | 71.4 ± 25.3 | 81.9 ± 20.2 | 81.9 ± 20.2 | 4.9 ± 0.1 |

All animals were necropsied at 20 days following the experimental M. bovis challenge. Lungs were removed and evaluated grossly for characteristic lesions attributable to a M. bovis infection. Percent lung damage scores are summarized in Table 5. Calves that were administered the experimental M. bovis challenge (Treatment Groups A, B, and C) had higher percent lung damage scores when compared to the non-challenged control animals (Treatment Group D). These results demonstrate that an experimental M. bovis challenge was capable of inducing characteristic lung lesions attributable to a M. bovis infection.

TABLE 5
Summary of Percent Lung Damage Scores
Mean Weighted Percentage ± Standard Deviation

| Treatment | |
|---|---|
| Group | Percent Lung Damage |
| A | 12.99 ± 8.13 |
| B | 11.06 ± 15.02 |
| C | 7.97 ± 5.96 |
| D | 0.71 ± 0.92 |

Each lung was lavaged with 50 ml of PBS. Results of the isolation of M. bovis from bronchial lavage samples twenty days following the experimental M. bovis challenge are summarized in Table 6. Calves that were administered the experimental M. bovis challenge (Treatment Groups A, B, and C) had an increase incidence of viable M. bovis in lung lavage samples when compared to the non-challenged control animals (Treatment Group D).

TABLE 6
Summary of Mycoplasma bovis Isolations from Lung Lavage Fluid

| Treatment Group | Number of Animals M. bovis Positive |
|---|---|
| A | 6/6 |
| B | 6/6 |
| C | 6/6 |
| D | 0/6 |

In conclusion, calves receiving the experimental M. bovis challenge (Treatment Groups A, B, and C) developed lung lesions, had increased rectal temperatures, decreased weight gain, and a increased incidence of viable M. bovis isolated from lung lavage samples when compared to the non-challenged control animals (Treatment Group D). The results show that the M. bovis culture was capable of inducing a serological response and was capable of inducing characteristic lung lesions attributable to a M. bovis infection following an experimental challenge.

### EXAMPLE 3

In this example, the M. bovis challenge model was evaluated in 5 to 7 month old calves. Twenty-two, healthy crossbred dairy or beef calves, approximately 4 weeks of age, with no maternal antibody to M. bovis were obtained and were randomly assigned by age as shown in Table 7. All calves were allowed to acclimatized prior to the initiation of the study.

TABLE 7
Experimental Treatment Groups

| Treatment | | Number |
|---|---|---|
| Group | Treatment | of Animals |
| A | Intranasal Challenge (3 consecutive days) | 14 |
| B | Non-Challenged Controls | 8 |

Calves were challenged as described above at approximately five to seven months of age. Calves in Group A received 20 ml (10 ml per nostril) of a fresh culture of M. bovis strain 5063 on three consecutive days and the challenge was administered with a simple spray device (Genesis Industries, Elmwood, Wis.). Animals in Group B were left as non-challenged control calves.

A viable count (CFU/ml) of the challenge inoculum was determined within one hour after the completion of each M. bovis experimental challenge. Results are shown in Table 8.

TABLE 8
Viable Count (CFU/ml) of Mycoplasma bovis Challenge Inoculum

| Challenge Culture | CFU/ml |
|---|---|
| Day 1 | 1.3 × 10⁸ |
| Day 2 | 4.0 × 10⁷ |
| Day 3 | 1.2 × 10⁸ |

All animals were weighed at 1 day prior to challenge and 21 days following experimental M. bovis challenge. Results are summarized in Table 9. Calves that were administered the experimental M. bovis challenge (Treatment Group A) had similar weight gains when compared to the non-challenged control animals (Treatment Group B).

TABLE 9
Summary of Body Weights Following Experimental Mycoplasma bovis
Challenge Mean Body Weight (kg) ± Standard Deviation

| Treatment | | | Weight |
|---|---|---|---|
| Group | Day -1 | Day 21 | Gain |
| A | 163.6 ± 32.61 | 169.6 ± 30.47 | 6.0 ± 6.56 |
| B | 162.5 ± 40.09 | 167.5 ± 35.15 | 5.0 ± 7.56 |

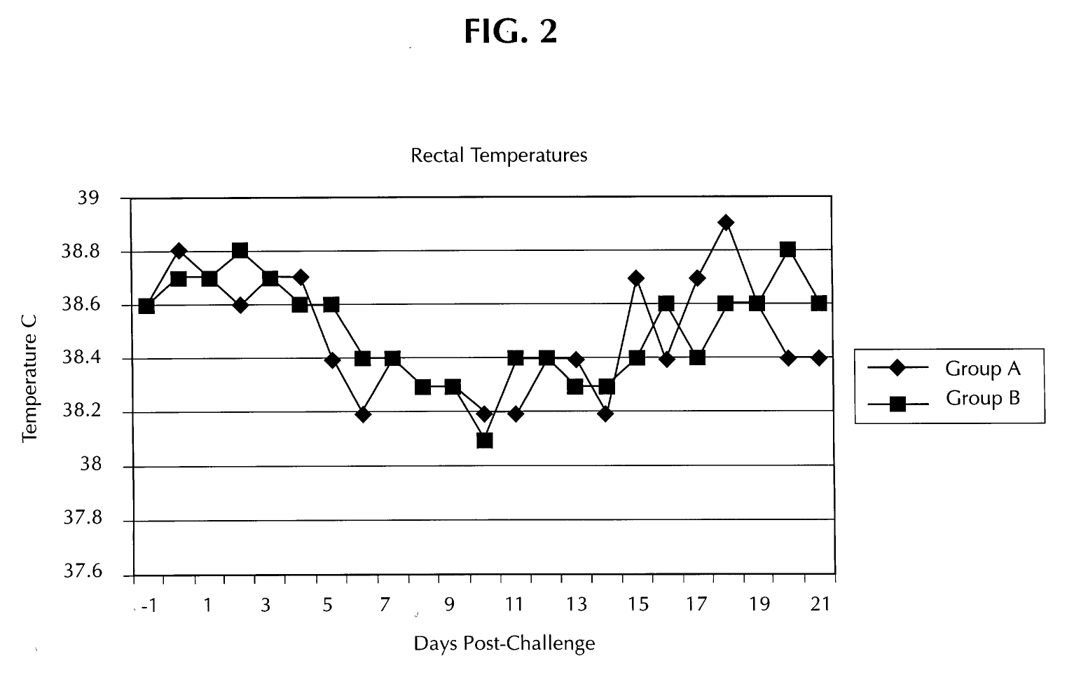

Rectal temperatures were measured each morning 1 day prior to challenge, immediately prior to challenge, and for 21 days following experimental M. bovis challenge. Results are summarized FIG. 2. M. bovis challenged calves (Treatment Group A) showed clinical signs, i.e. higher mean body temperatures on days 15, 17, and 18 when compared to the non-challenged control animals (Treatment Group B).

M. bovis specific serum antibody responses (IgG) are summarized in Table 10. Serum samples with mean percentage optical density (OD) values>80% of the positive control serum were considered positive for M. bovis. All calves were M. bovis negative prior to experimental challenge. Calves that received the experimental M. bovis challenge (Treatment Group A) were seropositive at 21 days following M. bovis challenge. Animals in Treatment Group B (non-challenged control animals) were negative at day 21.

TABLE 10
Summary of Mycoplasma bovis Serum Antibody (IgG)
Mean Percentage of Optical Density Values to Positive Control Serum
± Standard Deviation

| Treatment | | |
|---|---|---|
| Group | Day-1 | Day 21 |
| A | 27.9 ± 14.00 | 100.0 ± 0 |
| B | 26.5 ± 11.50 | 21.6 ± 19.2 |

All animals were necropsied at 21 days following the experimental M. bovis challenge. Lungs were removed and evaluated grossly for characteristic lesions attributable to a M. bovis infection. Percent lung damage scores are summarized in Table 11. Calves that were administered the experimental M. bovis challenge (Treatment Group A) had higher percent lung damage score when compared to the non-challenged control animals (Treatment Group B). These results demonstrate that an experimental M. bovis challenge was capable of inducing characteristic lung lesions attributable to a M. bovis infection.

TABLE 11
Summary of Percent Lung Damage Scores
Mean Weighted Percentage ± Standard Deviation

| Treatment | |
|---|---|
| Group | Percent Lung Damage |
| A | 11.10 ± 23.38 |
| B | 1.34 ± 2.77 |

Each lung was lavaged with 50 ml of PBS. Results of the isolation of M. bovis from bronchial lavage samples twenty-one days following the experimental M. bovis challenge are summarized in Table 12. Calves that were administered the experimental M. bovis challenge (Treatment Group A) had an increase incidence of viable M. bovis in lung lavage samples when compared to the non-challenged control animals (Treatment Group B).

TABLE 12
Summary of Mycoplasma bovis Isolations from Lung Lavage Fluid

| Treatment Group | Number of Animals M. bovis Positive |
|---|---|
| A | 10/14 |
| B | 0/8 |

In conclusion, calves receiving the experimental M. bovis challenge (Treatment Group A) developed lung lesions, had increased rectal temperatures on days 15, 17, and 18 following challenge, and a increased incidence of viable M. bovis isolated from lung lavage samples when compared to the non-challenged control animals (Treatment Group B). The results show that the M. bovis culture was capable of inducing a serological response and was capable of inducing characteristic lung lesions attributable to a M. bovis infection following an experimental challenge.

### EXAMPLE 4

In this example, three M. bovis strains were evaluated in young calves. Twenty-four, healthy crossbred dairy calves (Holstein/Friesian cross), approximately 6 weeks of age, with no maternal antibody to M. bovis were obtained and were randomly assigned by age as shown in Table 13. All calves were allowed to acclimatized for three weeks prior to the initiation of the study.

TABLE 13
Experimental Treatment Groups

| Treatment | | |
|---|---|---|
| Group | Treatment | Number of Animals |
| A | Intranasal Challenge strain 5063 | 6 |
| B | Intranasal Challenge strain 3625 | 6 |
| C | Intranasal Challenge strain 16150 | 6 |
| D | Non-Challenged Controls | 6 |

Calves were challenged as described above at approximately nine weeks of age. Each calf in Groups A, B, and C received 12 ml (6 ml per nostril) of a fresh culture of the appropriate M. bovis strain on three consecutive days and the challenge was administered with a simple spray device (Genesis Industries, Elmwood, Wis.). Animals in Group D were left as non-challenged control calves.

A viable count (CFU/ml) of each challenge inoculum was determined within one hour after the completion of each M. bovis experimental challenge. Results are shown in Table 14.

TABLE 14
Viable Count (CFU/ml) of Mycoplasma bovis Challenge Inoculum

| | Strain 5063 | Strain 3625 | Strain 16150 |
|---|---|---|---|
| Challenge | CFU/ml | CFU/ml | CFU/ml |
| Day 1 | 2.0 × 10⁸ | 1.0 × 10⁷ | 1.0 × 10⁷ |
| Day 2 | 2.0 × 10⁸ | 2.3 × 10⁸ | 2.0 × 10⁸ |
| Day 3 | 3.0 × 10⁸ | 5.0 × 10⁸ | 1.0 × 10⁸ |

All animals were weighed at 1 day prior to challenge and 21 days following experimental M. bovis challenge. Results are summarized in Table 15. Calves that were administered the experimental M. bovis challenge (Treatment Groups A, B, and C) had decreased weight gains when compared to the non-challenged control animals (Treatment Group D).

TABLE 15
Summary of Body Weights Following Experimental Mycoplasma bovis
Challenge Mean Body Weight (kg) ± Standard Deviation

| Treatment | | | Weight |
|---|---|---|---|
| Group | Day-1 | Day 21 | Gain |
| A | 73.4 ± 3.20 | 107.7 ± 7.86 | 34.3 ± 7.53 |
| B | 67.3 ± 5.77 | 89.1 ± 17.78 | 21.8 ± 15.30 |
| C | 69.3 ± 9.12 | 92.9 ± 10.5 | 23.6 ± 9.28 |
| D | 65.5 ± 9.89 | 104.2 ± 16.42 | 38.7 ± 7.93 |

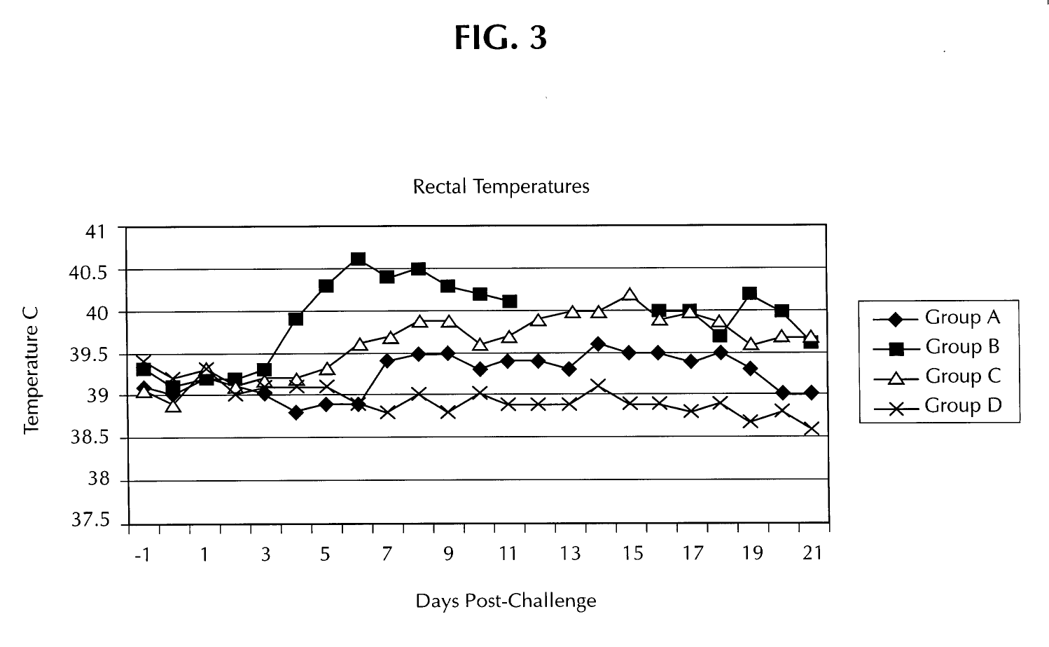

Rectal temperatures were measured each morning 1 day prior to challenge, immediately prior to challenge, and for 21 days following experimental M. bovis challenge. Results are summarized in FIG. 3 M. bovis challenged calves (Treatment Groups B and C) showed clinical signs, i.e. higher mean body temperatures on days 4 through 21 when compared to the non-challenged control animals (Treatment Group D). Group A challenged calves had higher mean body temperatures on days 7 through 21 when compared to Group D (non-challenged control animals).

M. bovis specific serum antibody responses (IgG) are summarized in Table 16. Serum samples with mean percentage optical density (OD) values >80% of the positive control serum were considered positive for M. bovis. All calves were M. bovis negative prior to experimental challenge. Calves that received a challenge culture of either M. bovis strain 5063, 3625, or 16150 (Treatment Groups A, B, and C) had a serological response at 20 days following the experimental challenge. Animals in Treatment Group D (non-challenged control animals) were negative at day 20.

TABLE 16
Summary of Mycoplasma bovis Serum Antibody (IgG)
Mean Percentage of Optical Density Values to Positive Control Serum
± Standard Deviation

| Treatment | | |
|---|---|---|
| Group | Day-1 | Day 21 |
| A | 0.98 ± 1.13 | 46.29 ± 14.23 |
| B | 0.43 ± 0.88 | 47.85 ± 28.50 |
| C | 0.77 ± 1.17 | 57.21 ± 48.24 |
| D | 0.98 ± 2.42 | 1.84 ± 1.74 |

All animals were necropsied at 21 days following the experimental M. bovis challenge. Lungs were removed and evaluated grossly for characteristic lesions attributable to a M. bovis infection. Percent lung damage scores are summarized in Table 17. Calves that were administered the experimental M. bovis challenges (Treatment Groups A, B, and C) had higher percent lung damage scores when compared to the non-challenged control animals (Treatment Group D). These results demonstrate that each M. bovis isolate was capable of inducing characteristic lung lesions attributable to a M. bovis infection.

TABLE 17
Summary of Percent Lung Damage Scores
Mean Weighted Percentage ± Standard Deviation

| Treatment | |
|---|---|
| Group | Percent Lung Damage |
| A | 8.65 ± 5.27 |
| B | 16.87 ± 27.91 |
| C | 32.36 ± 19.07 |
| D | 4.07 ± 3.01 |

### EXAMPLE 5

In this example, the efficacy of various M. bovis bacterins was evaluated in the M. bovis challenge model. Sixty-six, healthy crossbred dairy calves (Holstein/Friesian cross), approximately 14 days of age, with no maternal antibody to M. bovis were obtained and were randomly assigned by age. All calves were allowed to acclimate for one week prior to the initiation of the study.

The bacterins contained a BEI inactivated whole cell M. bovis bacteria at an appropriate concentration per dose. In addition, each vaccine preparation contained phosphate buffered saline (PBS) and an appropriate adjuvant (see Table 18.). The placebo contained PBS.

Animals were vaccinated with 2 ml of the appropriate vaccine or placebo by the subcutaneous route on day 0 (left neck) and on day 21 (right neck). The experimental treatment groups and vaccines used are shown in Table 19.

TABLE 18
Experimental Treatment Groups

| Treatment | | Number of |
|---|---|---|
| Group | Experimental Vaccines (2 ml dose) | Animals |
| A | M. bovis (5 × 10⁸ CFU) + Amphigen + Alhydrogel | 14 |
| B | M. bovis (5 × 10⁸ CFU) + Amphigen + QuilA/Cholesterol | 14 |
| C | M. bovis (5 × 10⁸ CFU) + Amphigen | 15 |
| D | Placebo (PBS) - Challenged Controls | 15 |
| E | Placebo (PBS) - Non-Challenged Controls | 8 |

Calves were challenged (day 42) as described above at approximately 9 weeks of age, 3 weeks following second vaccination. Animals in Groups A, B, C, and D were challenged with a broth culture by using a simple spray device (Genesis Industries, Elmwood, Wis.). Each calf received 12 ml (6 ml per nostril) of a fresh culture of M. bovis strain 5063 on three consecutive days. Animals in Group E were left as non-challenged control calves.

A viable count (CFU/ml) of each challenge inoculum was determined within one hour after the completion of each M. bovis experimental challenge. Results are shown in Table 19.

TABLE 19
Viable Count (CFU/ml) of Mycoplasma bovis Challenge Inoculum

| Challenge Culture | CFU/ml |
|---|---|
| Day 1 | 2.2 × 10⁹ |
| Day 2 | 3.2 × 10⁹ |
| Day 3 | 1.7 × 10⁹ |

All animals were weighed at 1 day prior to challenge, 7 days following challenge, 14 days following challenge, and 20 days following experimental M. bovis challenge. Results are summarized in Table 20. Calves that were administered the experimental M. bovis bacterins (Treatment Groups A, B, and C) and animals in Group E (non-challenged control animals) had increased weight gains when compared to the challenged control group (Treatment Group D).

TABLE 20
Summary of Body Weights Following Experimental Mycoplasma bovis Challenge
Mean Body Weight (kg) ± Standard Deviation

| Treatment | | | | | Weight |
|---|---|---|---|---|---|
| Group | Day 41 | Day 49 | Day 56 | Day 62 | Gain |
| A | 79.79 ± 12.29 | 88.00 ± 13.86 | 98.43 ± 12.35 | 103.71 ± 10.76 | 23.92 ± 5.99 |
| B | 78.21 ± 9.50 | 86.93 ± 9.90 | 98.29 ± 8.47 | 105.21 ± 9.32 | 27.00 ± 5.23 |
| C | 78.07 ± 16.78 | 86.60 ± 17.11 | 98.00 ± 20.92 | 104.00 ± 21.56 | 25.93 ± 8.80 |
| D | 78.93 ± 19.16 | 88.60 ± 20.44 | 94.43 ± 20.01 | 96.93 ± 20.89 | 18.00 |
| E | 81.38 ± 13.49 | 91.63 ± 13.00 | 105.00 ± 13.76 | 113.5 ± 15.94 | 32.12 ± 4.26 |

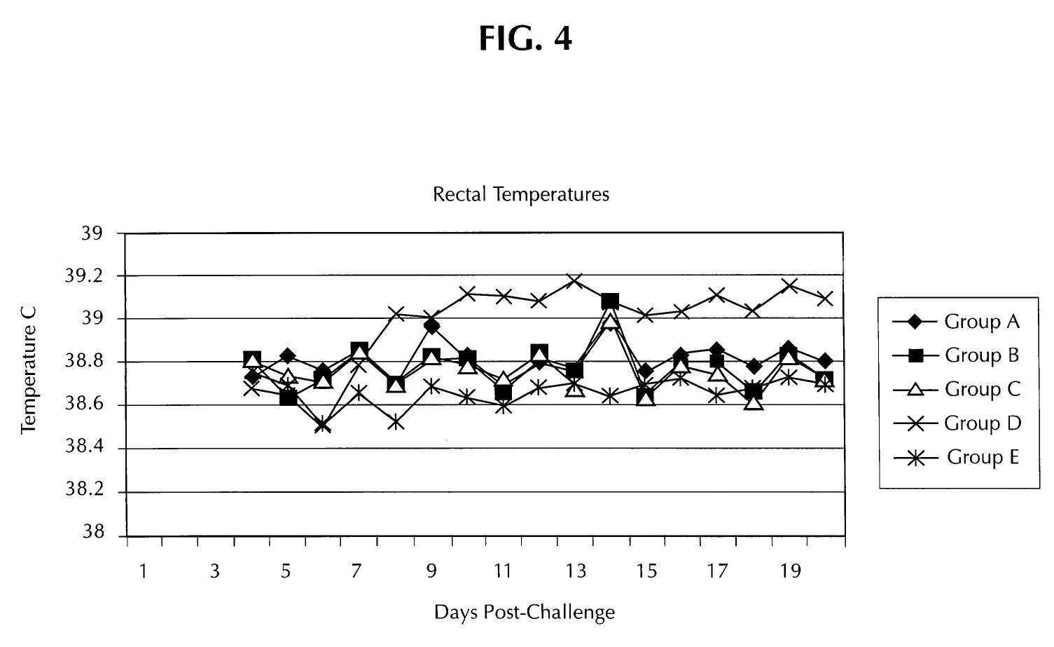

Please confirm the weight gain data for treatment group D. Rectal temperatures were measured each morning from three days to twenty days following experimental M. bovis challenge. Results are summarized in FIG. 4. Calves administered two doses of the M. bovis vaccines (Treatment Groups A and C) and animals in Group E (non-challenged control animals) showed clinical signs, i.e. lower mean body temperatures on days 7 through 20 when compared to the challenged control group (Treatment Group D). Vaccinated calves in Treatment Group B had lower mean body temperatures on days 7 to 12 and days 14 to 20 when compared to the challenged control group (Treatment Group D).

M. bovis specific serum antibody responses (IgG) are summarized in Table 21. Serum samples with mean percentage optical density (OD) values>80% of the positive control serum were considered positive for M. bovis. All calves were M. bovis negative prior to vaccination. Calves that received the experimental M. bovis bacterins (Treatment Groups A, B, and C) were seropositive to M. bovis prior to second vaccination and remained seropositive throughout the study. Animals in Treatment Group D (challenged control group) were seronegative until 20 days following the experimental M. bovis challenge. Calves in Treatment Group E (non-challenged control animals) were seronegative throughout the study.

TABLE 21
Summary of Mycoplasma bovis Serum Antibody (IgG)
Mean Percentage of Optical Density Values to Positive Control Serum ±
Standard Deviation

| Treatment | | | | | | |
|---|---|---|---|---|---|---|
| Group | Day −1 | Day 20 | Day 41 | Day 49 | Day 56 | Day 62 |
| A | Negative | 244.3 ± 66.0 | 314.7 ± 10.5 | 134.9 ± 7.4 | 115.5 ± 8.0 | 142.5 ± 6.9 |
| B | Negative | 262.1 ± 86.9 | 309.9 ± 33.6 | 139.5 ± 7.5 | 114.9 ± 7.5 | 145.0 ± 4.1 |
| C | Negative | 184.5 ± 60.6 | 292.2 ± 93.7 | 141.1 ± 9.1 | 118.9 ± 7.5 | 140.4 ± 7.7 |
| D | Negative | 36.9 ± 70.6 | 37.2 ± 81.0 | 37.4 ± 27.9 | 53.2 ± 39.4 | 100.5 ± 99.6 |
| E | Negative | 57.9 ± 58.3 | 46.9 ± 34.5 | 32.2 ± 22.1 | −3.3 ± 26.1 | 36.1 ± 21.6 |

All animals were necropsied at 20 days following the experimental M. bovis challenge. Lungs were removed and evaluated grossly for characteristic lesions attributable to a M. bovis infection. Percent lung damage scores and percent reduction of lung lesions are summarized in Table 22. Calves that were administered the experimental M. bovis bacterins (Treatment Groups A, B, and C) and animals in Group E (non-challenged control animals) had lower percent lung damage scores when compared to the challenged control animals (Treatment Group D). These results demonstrate that two doses of the experimental M. bovis bacterins were capable of inducing protection in calves following an experimental challenge.

TABLE 22
Summary of Percent Lung Damage Scores
Mean Weighted Percentage ± Standard Deviation

| Treatment | | |
|---|---|---|
| Group | Percent Lung Damage | Percent Reduction |
| A | 1.71 ± 3.03 | 77.5 |
| B | 1.49 ± 3.23 | 80.4 |
| C | 3.61 ± 6.17 | 52.5 |
| D | 7.60 ± 15.93 | — |
| E | 0.75 ± 1.02 | — |

Each lung was lavaged with 50 ml of PBS. Results of the isolation of M. bovis from bronchial lavage samples twenty days following the experimental M. bovis challenge are summarized in Table 23. Calves that were administered the experimental M. bovis bacterins (Treatment Groups A, B, and C) and animals in Group E (non-challenged control animals) had a reduced incidence and level of viable M. bovis in lung lavage samples when compared to the challenged control calves (Treatment Group D).

TABLE 23
Summary of Mycoplasma bovis Isolations from Lung Lavage Fluid

| | Number of Animals | |
|---|---|---|
| Treatment Group | M. bovis Positive | CFU/ml |
| A | 4/14 | 1.93 × 10² |
| B | 1/14 | 42.9 |
| C | 7/15 | 1.34 × 10⁶ |
| D | 12/15 | 4.50 × 10⁶ |
| E | 2/8 | 4.38 × 10² |

Please confirm the cfu/ml data for treatment group B. In conclusion, calves receiving the experimental M. bovis bacterins (Treatment Groups A, B, and C) and animals in Group E (non-challenged control animals) developed less lung lesions, had reduced rectal temperatures, increased weight gain, and a reduced level of viable M. bovis isolated from lung lavage samples when compared to the challenged control animals (Treatment Group D). The results show that two doses of the M. bovis bacterins were capable of inducing a serological response and protection from M. bovis in an experimental challenge model system.

1. A method for inducing a disease or disorder in an animal comprising administering an effective amount of a M. bovis culture and determining the clinical signs of said disease.

2. The method of claim 1 wherein said disease or disorder is selected from the group consisting of pneumonia, respiratory infections, lung lesions, arthritis, mastitis, otitis and reproductive disorders.

3. The method of claim 1 wherein said animal is selected from the group consisting of steer, bulls, cows, and calves.

4. The method of claim 3 wherein said animal is a cow.

5. The method of claim 3 wherein said animal is a calf.

6. The method of claim 1 wherein said effective amount of M. bovis culture comprises about 1×10⁶ to about 5×10¹¹ colony forming units (CFU) per challenge dose.

7. The method of claim 6 wherein said effective amount of M. bovis culture comprises about 1×10⁸ to about 1×10¹¹ CFU/dose.

8. The method of claim 7 wherein said effective amount of M. bovis culture comprises about 1×0¹⁰ to about 5×10¹⁰ CFU/dose.

9. The method of claim 1 wherein said clinical signs of disease comprise increased levels of M. bovis in the lung, increased temperatures, or decreased weight gains.

10. The method of claim 9 wherein said increased temperatures are rectal temperatures.

11. A method for assessing the efficacy of a vaccine against M. bovis comprising:
a. administering a M. bovis vaccine to a first animal;
b. challenging said animal with an effective amount of a M. bovis challenge culture;
c. challenging a second animal with an effective amount of a M. bovis challenge culture;
d. determining the clinical signs of a disease caused by said M. bovis; and
e. comparing the clinical signs of disease present in said first animal with the clinical signs of disease present in said second animal.

12. The method of claim 11 wherein said challenge culture comprises about 1×10⁶ to about 5×10¹⁰ colony forming units (CFU) per challenge dose.

13. The method of claim 12 wherein said challenge culture comprises about 1×10⁸ to about 1×10¹⁰ CFU/dose.

14. The method of claim 13 wherein said challenge culture comprises about 1×10¹⁰ to about 5×10¹⁰ CFU/dose.

15. The method of claim 11 wherein said clinical signs of disease comprise increased levels of M. bovis in the lung, increased temperatures, or decreased weight gains.
